(19) United States
(12) Patent Application Publication
(10) Pub. No.: US 2026/0224276 A1
(43) Pub. Date: Aug. 6, 2026

(54) END EFFECTOR WITH JAW SPACER AND RELATED METHODS

(71) Applicant: Cilag GmbH International, Zug (CH)

(72) Inventors: Austin E. Wise, Cincinnati, OH (US); Jeffrey L. Clark, Maineville, OH (US); John E. Brady, Liberty Township, OH (US); Matthew T. Stone, Mason, OH (US); Austin M. Fischer, Cincinnati, OH (US); Yajun Fan, Mason, OH (US)

(21) Appl. No.: 19/042,224

(22) Filed: Jan. 31, 2025

(51) Int. Cl. A61B 18/14; A61B 18/00

(52) U.S. Cl. CPC A61B 18/1445; A61B 2018/00077; A61B 2018/00083; A61B 2018/00184

(57) ABSTRACT

A surgical instrument with a jaw spacer and related methods includes a shaft assembly having a distal end and an end effector at the distal end of the shaft assembly. The end effector includes a first jaw and a second jaw. The first jaw has a first jaw body, and a first electrode surface secured relative to the first jaw body. The second jaw has a second jaw body, and an electrode assembly. The electrode assembly has a second electrode surface with the first and second electrode surfaces being operable to apply RF energy to tissue. The electrode assembly also has at least one electrical insulator interposed between the first electrode surface and the second electrode surface with the at least one electrical insulator being configured to separate the first electrode surface from the second electrode surface when in the closed configuration.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Further examples of such devices and related concepts are disclosed in U.S. Pat. No. 6,500,176 entitled “Electrosurgical Systems and Techniques for Sealing Tissue,” issued Dec. 31, 2002, the disclosure of which is incorporated by reference herein, in its entirety; U.S. Pat. No. 8,939,974, entitled “Surgical Instrument Comprising First and Second Drive Systems Actuatable by a Common Trigger Mechanism,” issued Jan. 27, 2015, the disclosure of which is incorporated by reference herein, in its entirety; U.S. Pat. No. 8,888,809, entitled “Surgical Instrument with Jaw Member,” issued Nov. 18, 2014, the disclosure of which is incorporated by reference herein, in its entirety; U.S. Pat. No. 9,161,803, entitled “Motor Driven Electrosurgical Device with Mechanical and Electrical Feedback,” issued Oct. 20, 2015, the disclosure of which is incorporated by reference herein, in its entirety; U.S. Pat. No. 9,877,720, entitled “Control Features for Articulating Surgical Device,” issued Jan. 30, 2018, the disclosure of which is incorporated by reference herein, in its entirety; U.S. Pat. No. 9,545,253, entitled “Surgical Instrument with Contained Dual Helix Actuator Assembly,” issued Jan. 17, 2017, the disclosure of which is incorporated by reference herein, in its entirety; and U.S. Pat. No. 9,526,565, entitled “Electrosurgical Devices,” issued Dec. 27, 2016, the disclosure of which is incorporated by reference herein, in its entirety.

Some electrosurgical instruments include an end effector with at least one insulating spacer. Examples of such instruments are described in U.S. Pat. No. 12,076,075, entitled “Electrosurgical Instrument with Floating Jaw Component,” issued Sep. 3, 2024, the disclosure of which is incorporated by reference herein, in its entirety.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4A depicts a side elevational view of a handle assembly of the electrosurgical instrument of FIG. 1, where the end effector is in an open and unfired state, where a portion of the handle assembly is omitted for purposes of clarity;

FIG. 4B depicts a side elevational view of the handle assembly of FIG. 4A, where the end effector is in a closed and unfired state, where a portion of the handle assembly is omitted for purposes of clarity;

FIG. 4C depicts a side elevational view of the handle assembly of FIG. 4A, where the end effector is in a closed and fired state, where a portion of the handle assembly is omitted for purposes of clarity;

FIG. 5A depicts a cross-sectional side view of the end effector of FIG. 2, where the end effector is in the open and unfired state, taken along line 5-5 of FIG. 2;

FIG. 5B depicts a cross-sectional side view of the end effector of FIG. 2, where the end effector is in the closed and unfired state, taken along line 5-5 of FIG. 2;

FIG. 5C depicts a cross-sectional side view of the end effector of FIG. 2, where the end effector is in the closed and fired state, taken along line 5-5 of FIG. 2;

FIG. 6 depicts a perspective view of another example of an end effector that may be incorporated into the electrosurgical instrument of FIG. 1;

FIG. 7 depicts a perspective view of an upper jaw of the end effector of FIG. 6;

FIG. 8 depicts a perspective view of a lower jaw of the end effector of FIG. 6;

FIG. 9 depicts an exploded perspective view of the end effector of FIG. 6;

FIG. 10 depicts a cross-sectional end view of the end effector of FIG. 6, taken about line 10-10 of FIG. 9;

FIG. 11 depicts a perspective view of another example of an end effector that may be incorporated into the electrosurgical instrument of FIG. 1, the end effector being shown in an open configuration;

FIG. 12 depicts a perspective view of the end effector of FIG. 11, the end effector being shown in a closed configuration;

FIG. 13 depicts a cross-sectional side view of the end effector of FIG. 11, the end effector being shown in the closed configuration with no tissue present;

FIG. 14 depicts a cross-sectional side view of the end effector of FIG. 11, the end effector being shown in the closed configuration with tissue present; and

FIG. 15 depicts an exploded perspective view of another example of an end effector.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

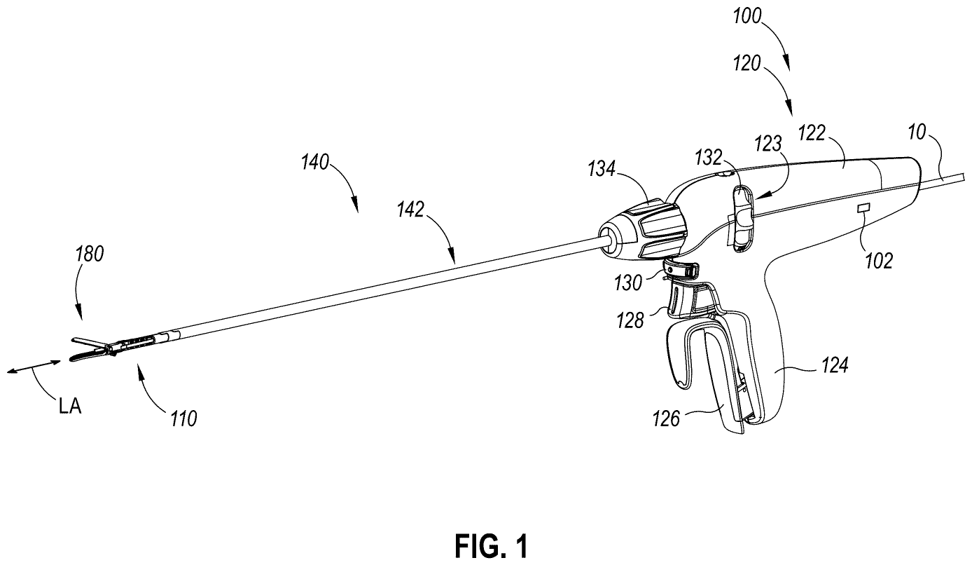

FIGS. 1-3C show an exemplary electrosurgical instrument (100). As best seen in FIG. 1, electrosurgical instrument (100) includes a handle assembly (120), a shaft assembly (140), an articulation assembly (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to seal or weld tissue by applying bipolar radio frequency (RF) energy to tissue. However, it should be understood electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a power source (not shown) via power cable (10).

The power source may be configured to provide all or some of the electrical power requirements for use of electrosurgical instrument (100). Any suitable power source may be used as would be apparent to one skilled in the art in view of the teachings herein. By way of example only, the power source may comprise a GEN04 or GEN11 sold by Ethicon Endo-Surgery, Inc. of Cincinnati, Ohio. In addition, or in the alternative, the power source may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to a power source via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrode surfaces (194, 196) of jaws (182, 184), respectively.

Body (122) of handle assembly (120) defines an opening (123) in which a portion of articulation control (132) protrudes from. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) is configured to bend articulation section (110) in order to drive deflection of end effector (180) from the longitudinal axis (LA) defined by shaft assembly (140). Articulation control (132) and articulation section (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

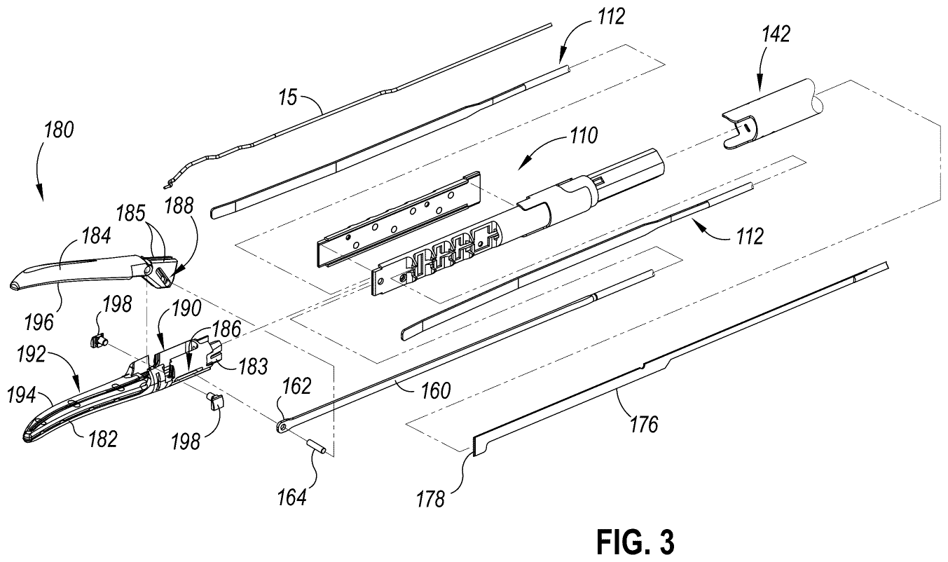

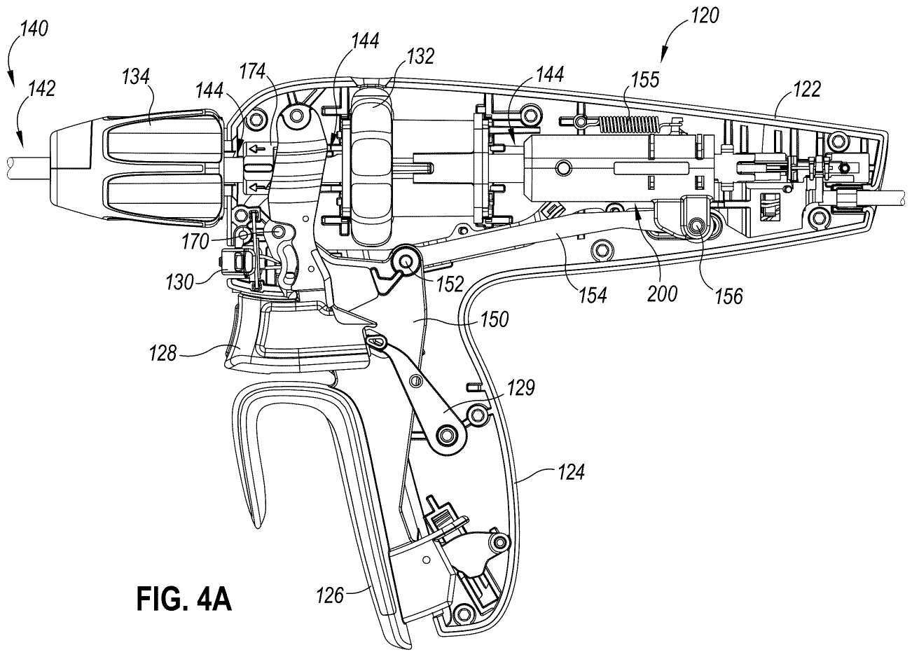

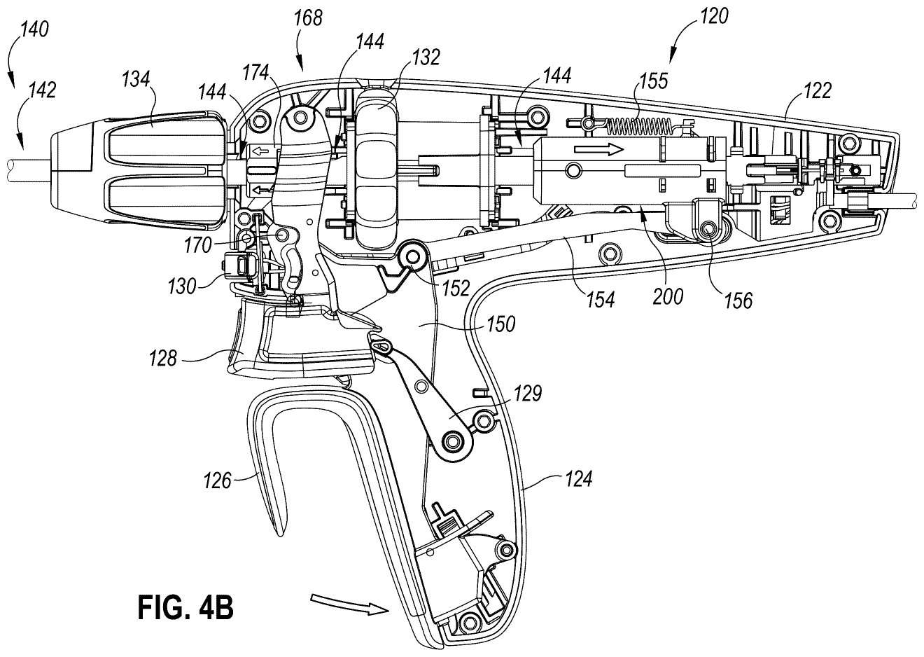

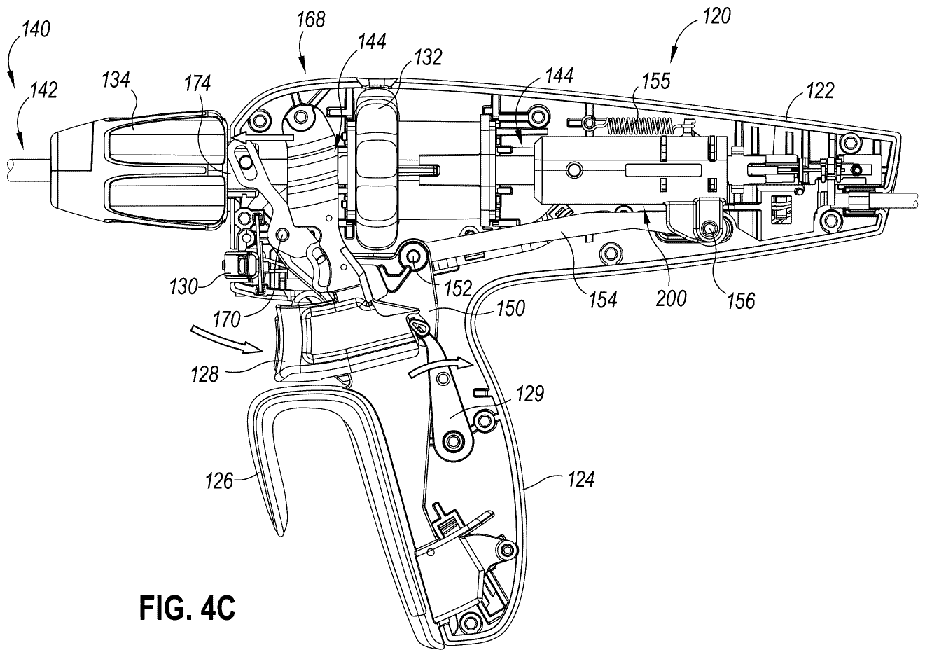

Shaft assembly (140) includes distal portion (142) extending distally from handle assembly (120), and a proximal portion (144) (see FIGS. 4A-4B) housed within the confines of body (122) of handle assembly (120). As best shown in FIG. 3, shaft assembly (140) houses a jaw closure connector (160) that couples jaw closure trigger (126) with end effector (180). Additionally, shaft assembly (140) houses a portion of knife member extending between distal cutting edge (178) and knife trigger (128). Shaft assembly (140) also houses actuating members (112) that couple articulation assembly (110) with articulation control (132); as well as an electrical connecter (15) that operatively couples electrode surfaces (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrode surface (194, 196).

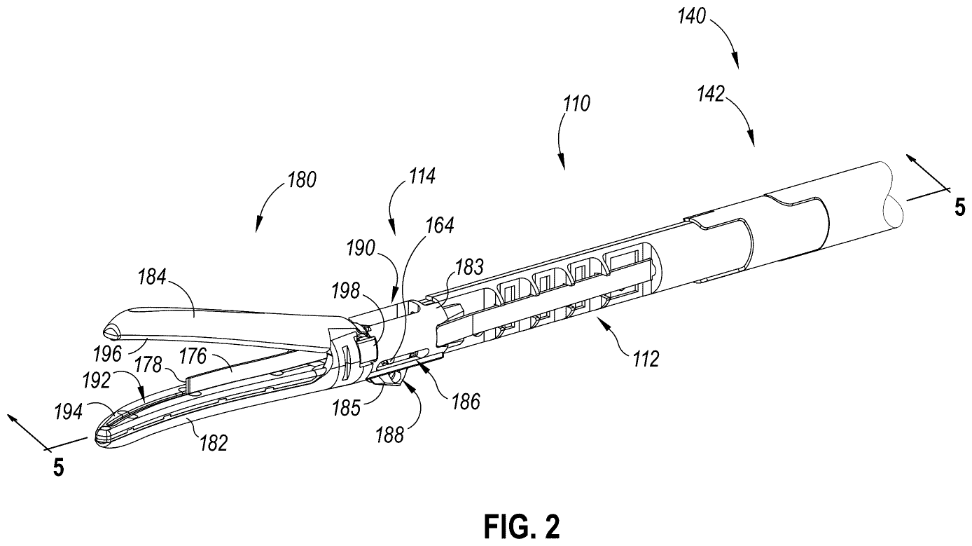

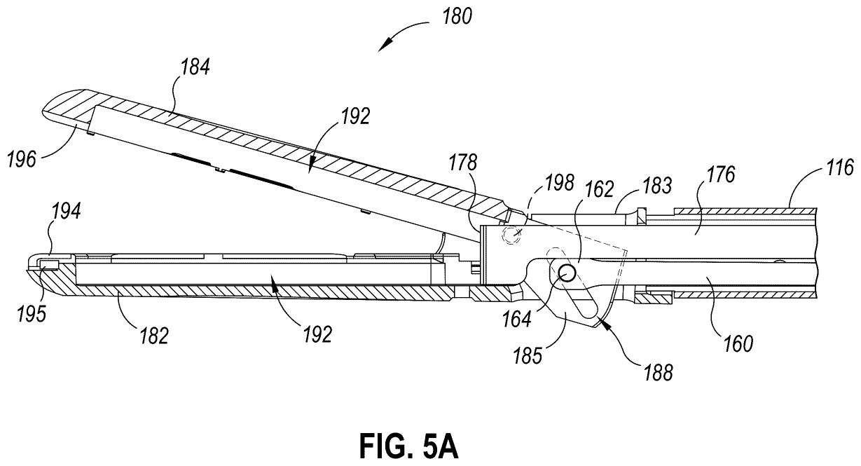

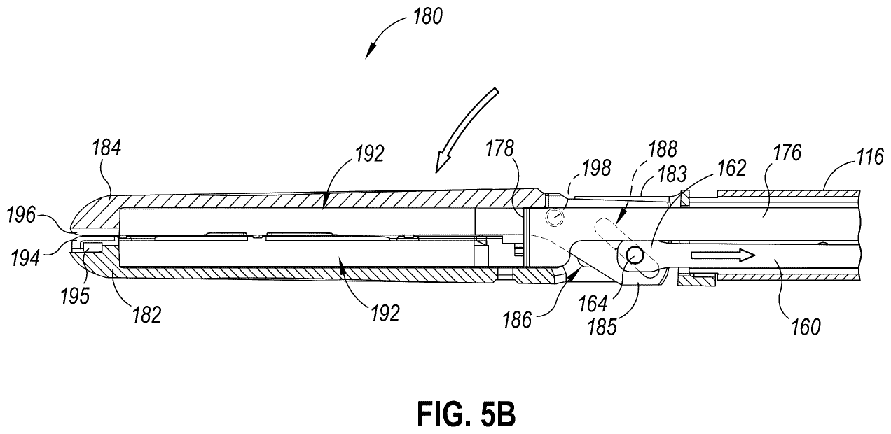

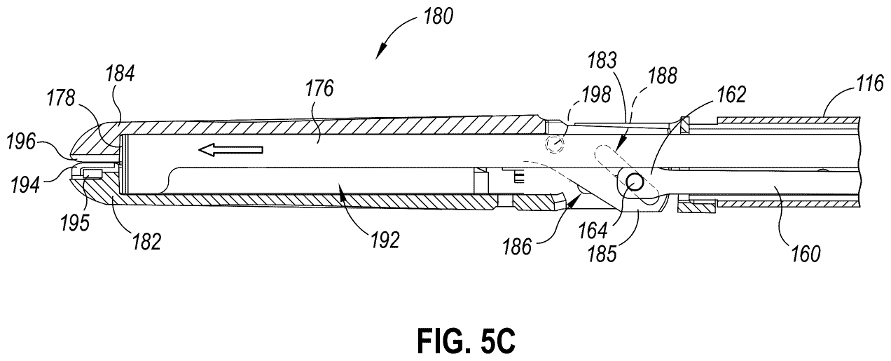

As best seen in FIGS. 2-3, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connecter (160), and pin (164). Slots (186, 188) each slidably receive pin (164), which is attached to a distal coupling portion (162) of jaw closure connector (160). Additionally, as best seen in FIGS. 5A-5C, lower jaw (182) includes a force sensor (195) located at a distal tip of lower jaw (182). Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge.

While in the current example, a force sensor (195) is incorporated into instrument (100) and is in communication with control unit (102), any other suitable sensors or feedback mechanisms may be additionally or alternatively incorporated into instrument (100) while in communication with control unit (102) as would be apparent to one skilled in the art in view of the teachings herein. For instance, an articulation sensor or feedback mechanism may be incorporated into instrument (100), where the articulation sensor communicates signals to control unit (102) indicative of the degree end effector (180) is deflected from the longitudinal axis (LA) by articulation control (132) and articulation section (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted (as shown in FIGS. 5A-5B), and advanced (as shown in FIG. 5C), to cut tissue captured between jaws (182, 184). Lower jaw (182) and upper jaw (184) each comprise a respective electrode surface (194, 196). The power source may provide RF energy to electrode surfaces (194, 196) via electrical coupling (15) that extends through handle assembly (120), shaft assembly (140), articulation assembly (110), and electrically couples with one or both of electrode surfaces (194, 196). Electrical coupling (15) may selectively activate electrode surfaces (194, 196) in response to an operator pressing activation button (130). In some instances, control unit (102) may couple electrical coupling (15) with activation button (130), such that control unit (102) activates electrode surfaces (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc.

FIGS. 4A-5C show an exemplary use of instrument (100) for end effector (180) to grasp, cut, and seal/weld tissue. As described above, and as shown between FIGS. 4A-4B and 5A-5B, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

Handle assembly (120) further includes a yoke assembly (200) that is slidably coupled along proximal portion (144) of shaft assembly (140). Yoke assembly (200) is operatively coupled with jaw closure connector (160) such that translation of yoke assembly (200) relative to proximal portion (144) of shaft assembly (140) translates jaw closure connector (160) relative to shaft assembly (140).

As best seen in FIGS. 4A-4C, yoke assembly (200) is coupled to a body (150) of jaw closure trigger (126) via a link (154). Link (154) is pivotally coupled with yoke assembly (200) via pin (156); while link (154) is also pivotally coupled with body (150) of jaw closure trigger (126) via pin (152). Additionally, jaw closure trigger (126) is pivotally coupled with body (122) of handle assembly (120) via pin (170). Therefore, as shown between FIGS. 4A-4B, an operator may pull jaw closure trigger (126) toward pistol grip (124), thereby rotating jaw closure trigger (126) about pin (170). Rotation of jaw closure trigger (126) leads to rotation of link (154) about both pins (152, 156), which in turn drives yoke assembly (200) in the proximal direction along proximal portion (144) of shaft assembly (140).

As described above, jaw closure connector (160) extends within shaft assembly (140), articulation section (110), and central channel (190) of lower jaw (182). As also mentioned above, jaw closure connector (160) is attached to pin (164). Therefore, as seen between FIGS. 5A-5B, proximal translation of yoke assembly (200) leads to proximal translation of pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

As best seen in FIGS. 4A-4C, yoke assembly (200) is also coupled with a bias spring (155). Bias spring (155) is also coupled to a portion of body (122), such that bias spring (155) biases yoke assembly (200) to the position shown in FIG. 4A (associated with the open configuration of end effector (180) as shown in FIG. 5A). Therefore, if an operator releases jaw closure trigger (126), bias spring (155) will translate yoke assembly (200) to the position shown in FIG. 4A, thereby opening jaws (182, 184) of end effector (180).

As described above, and as shown between FIGS. 4B-4C and 5B-5C, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body (174) that is slidably coupled along proximal portion (144) of shaft assembly (140). Knife coupling body (174) is coupled with knife member (176) such that translation of knife coupling body (174) relative to proximal portion (144) of shaft assembly (140) translates knife member (176) relative to shaft assembly (140).

As best seen in FIGS. 4B-4C and 5B-5C, knife coupling body (174) is coupled a knife actuation assembly (168) such that as knife trigger (128) pivots toward body (122) and/or pistol grip (124), knife actuation assembly (168) drives knife coupling body (174) distally, thereby driving knife member (176) distally within knife pathway (192). Because knife coupling body (174) is coupled to knife member (176), knife member (176) translates distally within shaft assembly (140), articulation section (110), and within knife pathway (192) of end effector (180), as best shown between FIGS. 5B-5C. Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

Knife trigger (128) is biased to the positions seen in FIGS. 4A-4B (associated with the knife member (176) in the retracted position) by a bias arm (129). Bias arm (129) may include any suitable biasing mechanism as would be apparent to one having ordinary skill in the art in view of the teachings herein. For instance, bias arm (129) may include a torsion spring. Therefore, if an operator releases knife trigger (128), bias arm (129) returns knife trigger (128) to the position shown in FIGS. 4A-4B, thereby translating knife member (176) toward the retracted position.

With distal cutting edge (178) of knife member (176) actuated to the advance position (position shown in FIG. 5C), an operator may press activation button (130) to selectively activate electrode surfaces (194, 196) of jaws (182, 184) to weld/seal severed tissue that is captured between jaws (182, 184). It should be understood that the operator may also press activation button (130) to selectively activate electrode surfaces (194, 196) of jaws (182, 184) at any suitable time during exemplary use. Therefore, the operator may also press activation button (130) while knife member (176) is retracted as shown in FIGS. 3A-3B. Next, the operator may release jaw closure trigger (128) such that jaws (182, 184) pivot into the opened configuration, releasing tissue.

### II. Example of End Effector with Floating Jaw Component

As mentioned above, end effector (180) is configured to grasp, sever, and weld/seal tissue. In particular, jaw (184) may pivot relative to jaw (182) in order to grasp tissue, while knife member (176) is configured to actuate within jaws (182, 184) in order to sever tissue that is grasped between jaws (182, 184). Electrode surfaces (194, 196) may be activated while jaws (182, 184) grasp tissue in order to weld/seal tissue captured between jaws (182, 184). While welding/sealing tissue captured between jaws (182, 184), an appropriate gap distance between electrode surfaces (194, 196) may be desirable along the entire length of electrode surfaces (194, 196). By way of example only, it may be desirable to provide a gap distance between electrode surfaces (194, 196) that ranges from approximately 0.002 inches to approximately 0.006 inches. If adjacent portions of electrode surfaces (194, 196) that cooperatively grasp tissue form a gap distance that is too small, tissue grasped between electrode surfaces (194, 196) may become crushed, damaged, etc. Additionally, if the gap distance is too small, electrode surfaces (194, 196) may come into incidental contact with each other to cause an undesirable short circuit. Conversely, if adjacent portions of electrode surfaces (194, 196) that cooperatively grasp tissue form a gap distance that is too large, electrode surfaces (194, 196) may not properly weld/seal tissue that is grasped between electrode surfaces (194, 196).

In some instances, the gap distance between electrode surfaces (194, 196) may deviate along the length of electrode surfaces (194, 196) such that a proximal portion of electrode surfaces (194, 196) form a gap distance of a first magnitude; and a distal portion of electrode surfaces (194, 196) form a gap distance of a second magnitude. In some such instances, the non-uniform gap distance along the length of electrode surfaces (194, 196) may be caused by deviations within manufacturing tolerances, such that different end effectors (180) provide different deviations in gap distances along the length of such end effectors (180) as a natural result of manufacturing processes. For instance, tolerances in the manufacture of jaws (182, 184), electrode surfaces (194, 196), slots (186, 188), pin (164), and/or other components may contribute to tolerance-related deviations from the desired gap distance between electrode surfaces (194, 196) and gap distance consistency along the length of electrode surfaces (194, 196).

Regardless of the cause, when the gap distance deviates along the length of an end effector (180), a first longitudinal region of electrode surfaces (194, 196) may create an acceptable tissue seal/weld, while a second longitudinal region of electrode surfaces (194, 196) may have too small a gap distance that may cause undesirable effects as mentioned above. It may therefore be desirable to provide a form of end effector (180) that accommodates manufacturing tolerances and reliably provides a gap distance that achieves the desired effects along the entire length of electrode surfaces (194, 196). In other words, it may be desirable to provide a modified form of end effector (180) that includes one or more features to ensure a minimum gap distance between opposing electrodes. An example of such an alternative form of end effector (180) is described in greater detail below.

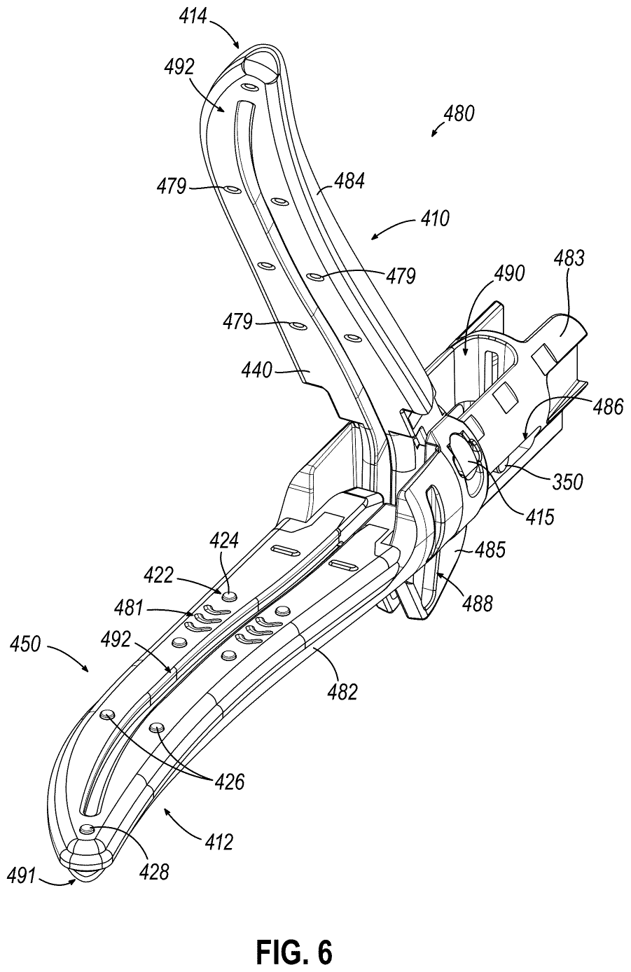

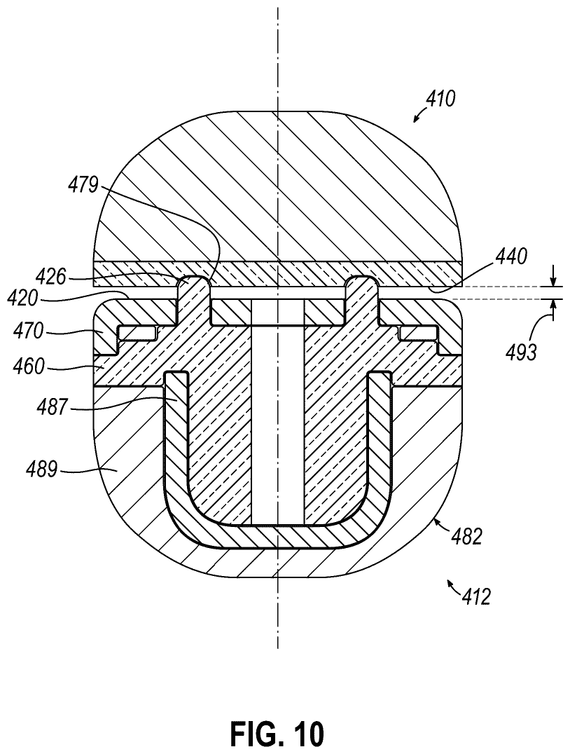

FIG. 6 shows an example of another end effector (480) that may be readily incorporated into electrosurgical instrument (100) in replacement of end effector (180) described above. End effector (480) is substantially similar in form and function to end effector (180) described above, with differences elaborated below. As will be described in greater detail below, end effector (480) includes a lower electrode assembly (450) that is configured to maintain a fixed gap (493)(see FIG. 10) and thereby promote an acceptable weld/seal of tissue grasped by end effector (480) in accordance with the description herein.

End effector (480) of the present example includes an upper jaw (410) and a lower jaw (412). Except for the differences described in greater detail below, upper jaw (410) and lower jaw (412) may be configured and operable like upper jaw (184) and lower jaw (182), respectively, described above. Upper jaw (410) is configured to pivot relative to lower jaw (412) in order to grasp tissue between jaws (410, 412). As will described in detail below, an electrode surface (420) includes tissue grasps (481) and electrode surface (440) include electrode recesses (479).

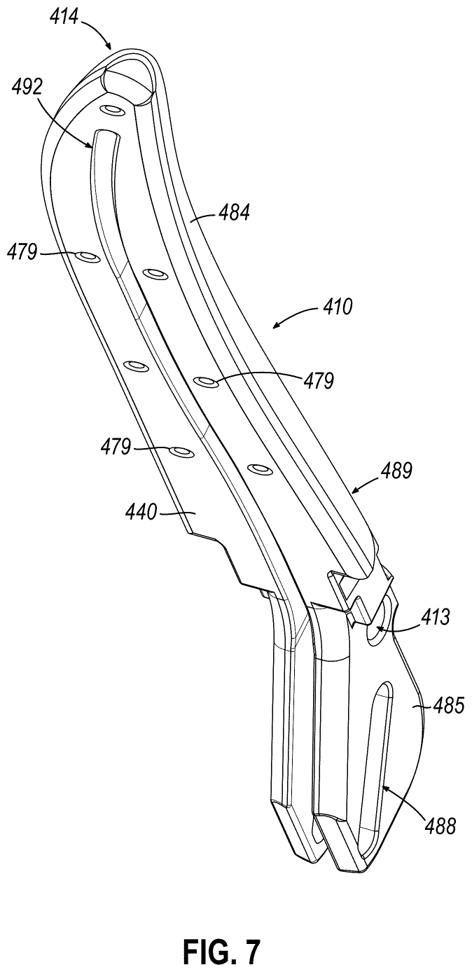

FIG. 7 shows upper jaw (410) with an upper jaw body (484) and a pair of proximal arms (485) that extend proximally from upper jaw body (484). Proximal arms (485) may be configured and operable like proximal arms (185) described above. Proximal arms (485) define a slot (488), which is configured and operable like slot (188) described above. Slot (488) is configured to receive a pin (350), which is operable to drive pivotal movement of upper jaw (410) relative to lower jaw (412) as will be described in greater detail below. An aperture (413) in upper jaw (410) is configured to receive another pin (415), which is configured to provide a pivot axis as will be described in greater detail below.

Upper jaw (410) further includes an electrode surface (440). Electrode surface (440) may be configured and operable like electrode surface (196) described above. Electrode surface (440) may thus be coupled with a power source such that the power source may provide RF energy to electrode surface (440). An operator may thus selectively activate electrode surface (440) by pressing activation button (130) in accordance with the description above. Electrode surface (440) includes electrode recesses (479) sized and shaped to fit teeth (424, 426, 428) of ceramic body (460), as discussed below.

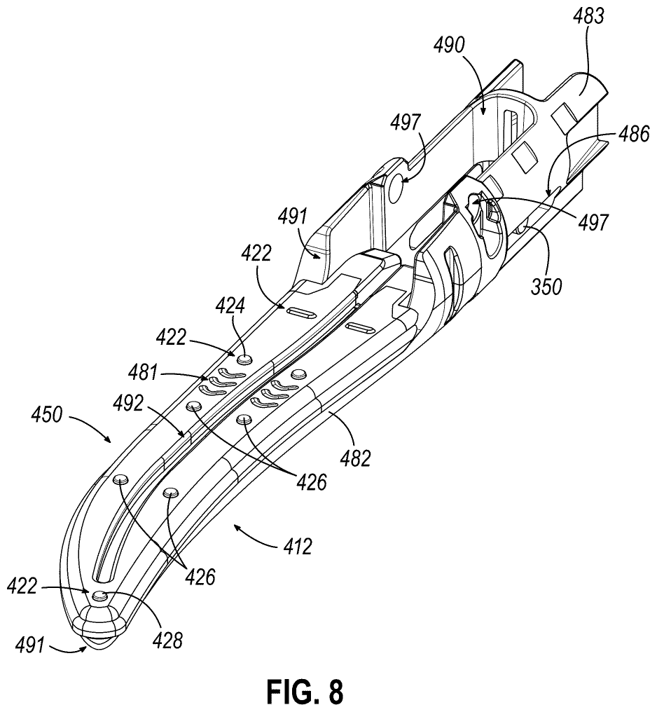

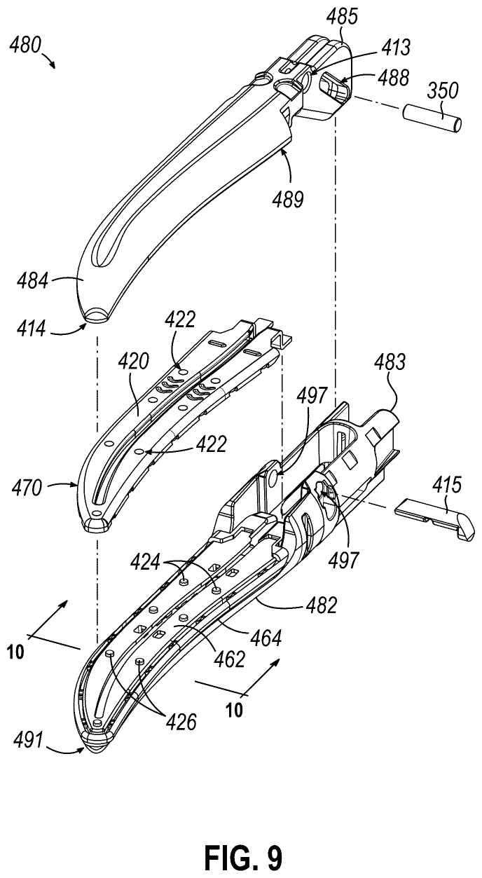

As best shown FIGS. 8-9, lower jaw (412) includes a lower jaw body (482) and a proximal body (483) that extends proximally from lower jaw body (482). Proximal body (483) may be configured and operable like proximal body (183) described above. Proximal body (483) defines a central channel (490) and a slot (486), which are respectively configured and operable like central channel (190) and slot (186) described above. Slot (486) of lower jaw (412) is configured to receive the same pin (350) as slot (488) of upper jaw (410). As noted above, this pin (350) is operable to drive pivotal movement of upper jaw (410) relative to lower jaw (412) as described above in the context of FIGS. 5A-5B. Pivotal movement of upper jaw (410) toward and away from lower jaw (412) is driven via translation of pin (350) within slots (486, 488). Pin (350) of this example is thus configured and operable like pin (164) described above. Proximal body (483) further defines an aperture (197) that is configured to receive the same pin (415) as aperture (413) of upper jaw (410). Pin (415) is configured to provide a pivot axis for the pivotal movement of upper jaw (410) relative to lower jaw (412). As noted above, upper jaw (410) is configured to pivot relative to lower jaw (412) in order to grasp tissue between jaws (410, 412).

Lower jaw (412) and upper jaw (410) further define a knife pathway (492) that is dimensioned to slidably receive knife member (176) when jaws (410, 412) are in a closed configuration in accordance with the description herein (e.g., as described above with reference to FIGS. 5B-5C).

As best seen in FIG. 9, lower jaw (412) of the present example further includes an electrode assembly (450). As best seen in FIG. 9, electrode assembly (450) includes a ceramic body (460) and an electrode body (470). While ceramic is used to form body (460) in the present example, any other suitable electrically insulative material(s) may be used. Ceramic body (460) includes an upper surface (462), an outer edge (464), and a plurality of teeth (424, 426, 428). This plurality of teeth (424, 426, 428) includes a pair of proximal teeth (424), two pairs of middle teeth (426), and a distal tooth (428). While in the current example, there are four pairs of teeth (424, 426) and a distal tooth (428) longitudinally spaced apart from each other, any suitable number of teeth (424, 426, 428) in any suitable array/pattern may be used as would be apparent to one skilled in the art in view of the teachings herein.

Electrode body (470) of the present example is configured to fit atop ceramic body (460). Electrode body (470) may be coupled to ceramic body (460) in any suitable fashion, such as via an adhesive, an interference fit, a snap fit, a latch, etc. Electrode body (470) includes an electrode surface (420) defining a plurality of apertures (422). Similar to electrode surface (194) described above, electrode surface (420) may be coupled with a power source such that the power source may provide RF energy to electrode surface (420). An operator may thus selectively activate electrode surface (420) by pressing activation button (130) in accordance with the description above. Electrode surface (420) of lower jaw (412) may cooperate with electrode surface (440) of upper jaw (410) to apply bipolar RF energy to tissue that is captured between electrode surface (420) and electrode surface (440).

Each tooth (424, 426, 428) of ceramic body (460) is received in a respective aperture (422) defined by electrode surface (420), such that each tooth (424, 426, 428) passes through its respective aperture (422) and protrudes past electrode surface (420). Since teeth (424, 426, 428) are formed by ceramic body (460) in this example, teeth (424, 426, 428) are electrically insulated from electrode surface (420) while also extending above the electrode surface (420). Therefore, if any tooth (424, 426, 428) comes into contact with electrode surface (440) of upper jaw (410), such contact will not result in a short circuit.

In the current example, teeth (424, 426, 428) have different heights relative to electrode surface (420). In particular, proximal teeth (424) and distal tooth (428) are taller than middle teeth (426), such that proximal teeth (424) and distal tooth (428) extend past electrode surface (420) further than middle teeth (426). In addition, distal tooth (428) is taller than proximal teeth (424). In some instances, a height disparity among teeth (424, 426, 428) may be intentional. In some other instances, such height disparity may be the unintentional result of irregularities within tolerance in the manufacturing process.

By way of example only, the configuration of distal tooth (428) may promote use of distal tooth (428) to grip tissue as the tissue is being compressed between jaws (410, 412). In addition, or in the alternative, the configuration of distal tooth (428) may prevent tissue from being “milked” or squeezed distally out from jaws (410, 412) as jaws (410, 412) transition to a closed state to compress the tissue. In addition, or in the alternative, the configuration of distal tooth (428) may allow distal tooth (428) to govern the tissue gap at distal ends (414, 491). For instance, as jaws (410, 412) transition to the closed state, distal tooth (428) may contact electrode surface (440) near distal end (414) and thereby prevent distal end (414) from moving further toward distal end (491), even if the operator continues to further pivot closure trigger (126) toward pistol grip (124) after distal tooth (428) makes initial contact with electrode surface (440).

By way of further example only, the configuration of proximal teeth (424) may allow proximal teeth (424) to firmly grip tissue as knife member (174) is driven through knife pathway (492) as knife member (174) is driven distally while the tissue is being compressed between jaws (410, 412). In other words, with proximal teeth (424) firmly gripping the tissue, there may be little to no risk that knife member (174) will push the tissue distally as knife member (174) is driven distally while the tissue is being compressed between jaws (410, 412). In versions where proximal teeth (424) are configured to be taller than middle teeth (426), this greater height of proximal teeth (424) may increase the likelihood of proximal teeth (424) engaging tissue to a desirable extent near proximal end (468) of ceramic body (466). In scenarios where manufacturing variations provide a gap distance between electrode surfaces (420, 440) that is unacceptable along the length of end effector (480), such deviations may also provide unacceptable distances between proximal teeth (424) and upper jaw (410).

FIG. 10 shows a cross section of lower jaw (412) taken about line 10-10. Above mentioned lower jaw body (482) includes a lower jaw overmold (489) wrapped around a lower jaw channel (487). Lower jaw channel (487) may be metal and formed via a stamping process. As a metal, lower jaw channel (487) may be conductive. In order to prevent energy from electrode (470) from passing to lower jaw channel (487), ceramic body (460) may be positioned between electrode (470) and lower jaw channel (487). Lower jaw overmold (489) may then be formed around portions of lower jaw channel (487) and ceramic body (460). Any of teeth (424, 426, 428) may then extend from ceramic body (460), towards upper jaw (410), such that teeth (424, 426, 428) extend beyond electrode (470). Any of teeth (424, 426, 428) may be of a bulbous shape such a spherical half dome. Lower jaw overmold (489) may be beneficial to guide end effector (480) during positioning in the patient and to electrically insulate patient from portions of electrode (470). Lower jaw overmold (489) may also be beneficial to adhere or secure electrode (470) to ceramic body (460) and ceramic body (460) to lower jaw channel (487). Ceramic body (460) may extend to a lateral side of lower jaw (412), as shown in FIG. 10.

As shown, previously mentioned recesses (479) may be formed and positioned such that they engage with respective teeth (424, 426, 428). Recesses (479) may be similarly shaped and sized to respective teeth (424, 426, 428) such that they provide a close fit. They may alternatively be oversized to thereby allow for any slight misalignment between jaws (410, 412) when in the closed configuration. Recesses (479) may be larger that teeth (424, 426, 428) and may act as a guide for transitioning jaws (410, 412) from the open configuration to the closed configuration. Recesses (479) may be formed directly into electrode (440) via a coining, stamping, machining, or metal etch Process.

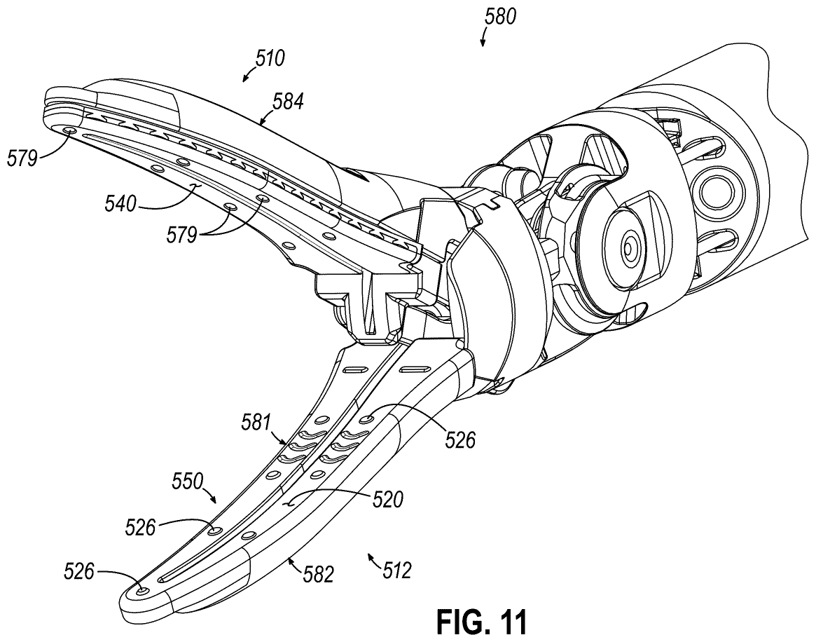

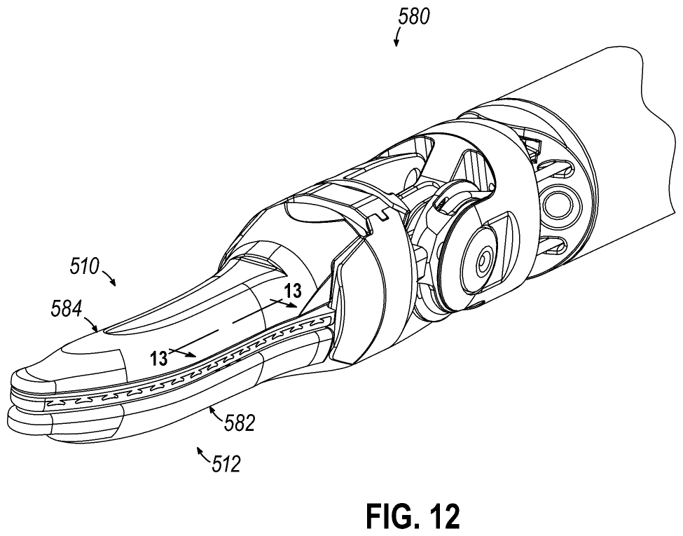

FIGS. 11-12 show an example of another end effector (580) that may be readily incorporated into electrosurgical instrument (100) in replacement of end effectors (180, 480) described above. End effector (580) is substantially similar in form and function to end effectors (180, 480) described above, with differences elaborated below. As will be described in greater detail below, end effector (580) includes a lower electrode assembly (550) that is configured to maintain a fixed gap and thereby promote an acceptable weld/seal of tissue grasped by end effector (480) in accordance with the description herein.

End effector (580) of the present example includes an upper jaw (510) and a lower jaw (512). Upper jaw (510) includes an upper jaw body (584) while lower jaw (512) includes lower jaw body (582). Except for the differences described in greater detail below, upper jaw (510) and lower jaw (512) may be configured and operable like upper jaws (184, 410) and lower jaw (182, 412), respectively, described above. Upper jaw (510) is configured to pivot relative to lower jaw (512) in order to grasp tissue between jaws (510, 512). As will described in detail below, an electrode surface (520) includes tissue grasps (581) and electrode surface (594) includes electrode recesses (579).

Electrode surface (520) includes teeth (526) which can be adhered to the top of electrode surface (520) using an adhesive or via a thermal spray. Teeth (526) are electrically insulating to thereby prevent an electrical short between electrode surfaces (520, 540) by maintaining a predefined gap. Teeth (526) are raised and interposed between electrode surfaces (520, 540) and may be of a ceramic material, similar to teeth (426). Tissue grasps (581) may be positioned longitudinally between teeth (426) as shown and may protrude from electrode surface (520) less than teeth (526). As such, a lesser gap (595) may be formed between tissue grasps (581) and electrode surface (540) than a gap (593) between electrode surfaces (520, 540). Tissue grasps (581) may be formed into and integral with electrode surface (520) to thereby be electrically conductive and thus transmit energy between lower jaw (512) and upper jaw (510). Alternatively, tissue grasps (581) may be electrically insulating and adhered to electrode surface (520), similar to teeth (526). Tissue grasps (581) may be arched shaped or wave shaped and may be wider than a width of teeth (526). Tissue grasps (581) can be used to secure tissue between upper jaw (510) and lower jaw (512). FIG. 12 shows end effector (580) in the closed orientation such that electrode surfaces (520, 540) are spaced apart by a gap.

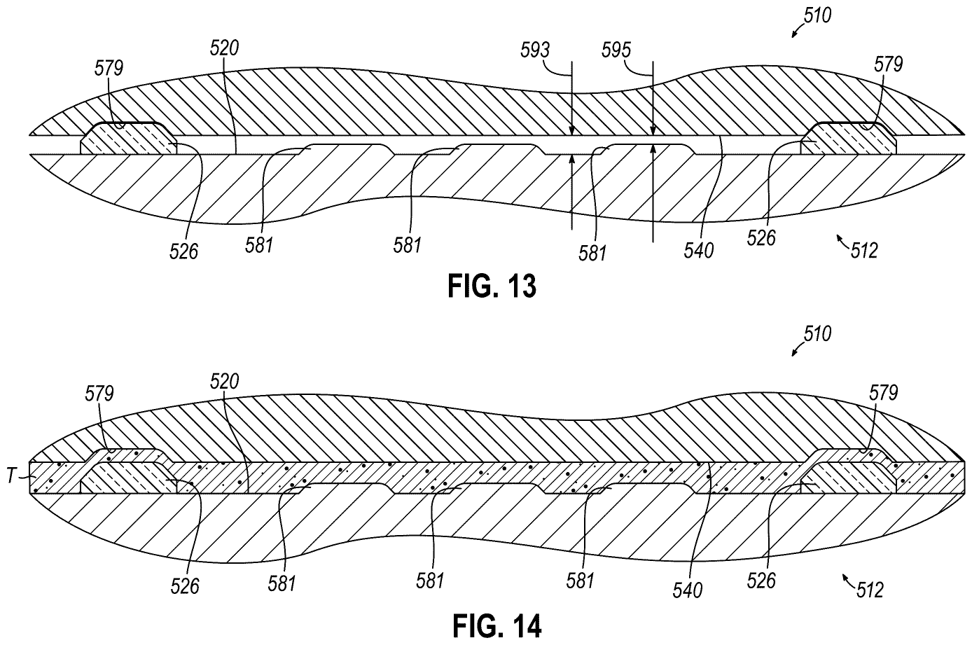

FIGS. 13-14 show electrode surfaces (520, 540) both without (FIG. 13) and with (FIG. 14) tissue interposed between. As shown in FIG. 13, when no tissue is present, teeth (526) act to separate electrode surfaces (520, 540). As described above and as with recesses (479), recesses (579) are sized and shaped to fit teeth (526). Recesses (579) may act as a guide for teeth (526) while end effector (580) transitions from the open configuration to the closed configuration. As shown, recesses (579) are larger than teeth (526) and include a flat top and straight edges. Other embodiments can include recesses (579) where the size and/or shape mirrors teeth (526). Recesses (579) may be formed into electrode surface (540) via a stamping, coining, machining, chemical etch, or any other process reasonably foreseeable by one skilled in the art.

FIG. 14 shows tissue present between electrode surfaces (520, 540) such that tissue is present inside recesses (579) and that electrode surfaces (520, 540) are separated by an even greater gap when in the closed configuration. Accordingly, tissue is restrained by teeth (526), recesses (579), and tissue grasps (581).

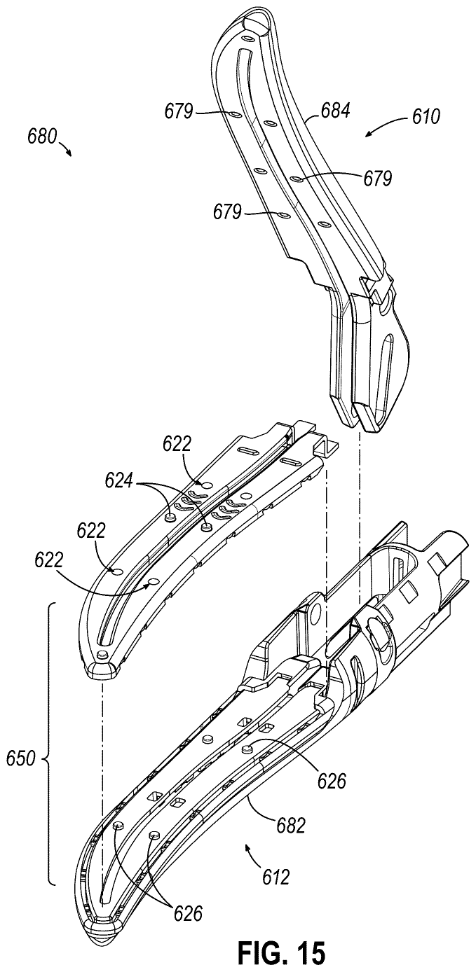

FIG. 15 shows an end effector (680) including aspects of each of end effectors (180, 480, 580). Accordingly, end effector (680) may be substantially similar in form and function to end effectors (180, 480, 580). Specifically, end effector (680) includes an upper jaw (610) having an upper jaw body (684) affixed relative to an electrode surface (640) having electrode recesses (679). End effector (680) also includes a lower jaw (612) having a lower jaw body (682) affixed relative to a lower electrode assembly (650) having both apertures (622) and teeth (624). Teeth (624) may be substantially similar in form and function to teeth (526). Protruding from apertures (622) are teeth (626) which may be substantially similar in form and function to teeth (426).

While upper jaw (610) is shown including recesses (679) and lower jaw (612) is shown including teeth (624, 626) and apertures (622), it is envisioned that these features may be switched or mixed between jaws such that each jaw includes a combination of recesses (679), teeth (624, 626), and apertures (622).

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: a shaft assembly having a distal end; and an end effector at the distal end of the shaft assembly, the end effector including: a first jaw, including: a first jaw body, and a first electrode surface secured relative to the first jaw body, and a second jaw movably coupled relative to the first jaw and configured to selectively move from an open configuration toward a closed configuration, the second jaw including: a second jaw body, and an electrode assembly secured relative to the second jaw body, the electrode assembly including: a second electrode surface positioned to face the first electrode surface when the first and second jaws are placed in the closed configuration, the first and second electrode surfaces being operable to apply RF energy to tissue, and at least one electrical insulator interposed between the first electrode surface and the second electrode surface, the at least one electrical insulator being configured to separate the first electrode surface from the second electrode surface when in the closed configuration, the at least one electrical insulator being rigidly affixed relative to the second jaw body.

### Example 2

The surgical instrument of Example 1, wherein the at least one electrical insulator is raised away from the second electrode surface and toward the first electrode surface.

Example 3

The surgical instrument of any one or more of Examples 1 through 2, wherein the at least one electrical insulator is securely affixed to the second electrode surface.

### Example 4

The surgical instrument of any one or more of Examples 1 through 3, wherein the at least one electrical insulator is configured to restrict a movement of the tissue between the first and second electrode surfaces when in the closed configuration.

### Example 5

The surgical instrument of any one or more of Examples 1 through 4, wherein the first electrode surface includes at least one recess, wherein the at least one electrical insulator is sized to nest within the at least one recess when the first and second jaws are placed in the closed configuration.

### Example 6

The surgical instrument of Example 5, wherein the at least one recess has a different shape than the at least one electrical insulator.

### Example 7

The surgical instrument of any one or more of Examples 1 through 6, wherein the at least one electrical insulator includes a first electrical insulator and a second electrical insulator, wherein the first electrical insulator is distally positioned relative to the first electrical insulator.

### Example 8

The surgical instrument of Example 7, wherein at least one of the first electrode surface and the second electrode surface includes a tissue grasper positioned between the first and second electrical insulators.

### Example 9

The surgical instrument of Example 8, wherein the tissue grasper protrudes from the second electrode surface less than the first electrical insulator protrudes from the second electrode surface.

### Example 10

The surgical instrument of Example 9, wherein the tissue grasper is electrically conductive.

### Example 11

The surgical instrument of any one or more of Examples 1 through 10, wherein the second jaw further includes an insulating spacer positioned on the second electrode surface opposite from the first electrode surface, and wherein the insulating spacer and the at least one electrical insulator are integrally formed.

### Example 12

The surgical instrument of Example 11, wherein the second jaw further includes a channel affixed to the insulating spacer such that the insulating spacer is positioned between the channel and the first electrode surface.

### Example 13

The surgical instrument of any one or more of Examples 1 through 12, wherein the at least one electrical insulator includes a ceramic material.

### Example 14

The surgical instrument of any one or more of Examples 1 through 13, wherein the at least one electrical insulator is affixed along a flat portion the second electrode surface such that the flat portion extends beyond the at least one electrical insulator.

### Example 15

The surgical instrument of any one or more of Examples 1 through 14, wherein the at least one electrical insulator is configured to contact the first electrode surface.

### Example 16

A surgical instrument, comprising: a shaft assembly having a distal end; and an end effector at the distal end of the shaft assembly, the end effector including: a first jaw, including: a first jaw body, and a first electrode surface secured relative to the first jaw body, and a second jaw pivotably coupled with the first jaw and configured to selectively move from an open configuration toward a closed configuration, the second jaw including: a second jaw body having a distal end, and an electrode assembly coupled with the second jaw body, the electrode assembly including: a second electrode surface positioned to face the first electrode surface when the first and second jaws are placed in the closed configuration, the first and second electrode surfaces being operable to apply RF energy to tissue, and at least one electrical insulator interposed between the first electrode surface and the second electrode surface, the at least one electrical insulator being configured to separate the first electrode surface from the second electrode surface when in the closed configuration, the at least one electrical insulator being rigidly affixed relative to the second jaw body.

### Example 17

A method of assembling a surgical instrument, comprising: pivotally securing a first jaw to a second jaw, the first jaw including a first electrode surface, the second jaw including a second electrode surface, the first and second electrode surfaces being operable to apply RF energy to tissue; securing at least one electrical insulator along the second electrode surface; and separating the first and second electrode surfaces from each other via at least one electrical insulator.

### Example 18

The method of Example 17, further comprising nesting the at least one electrical insulator within an at least one recess of the first electrode surface.

### Example 19

The method of Example 18, further comprising forming the at least one recess using a coining and/or photo-chemical machining process.

### Example 20

The method of any one or more of Examples 1 through 17, further comprising grasping the tissue with the at least one electrical insulator.

### IV. Miscellaneous

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument, comprising:
(a) a shaft assembly having a distal end; and
(b) an end effector at the distal end of the shaft assembly, the end effector including:
(i) a first jaw, including:
(A) a first jaw body, and
(B) a first electrode surface secured relative to the first jaw body, and
(ii) a second jaw movably coupled relative to the first jaw and configured to selectively move from an open configuration toward a closed configuration, the second jaw including:
(A) a second jaw body, and
(B) an electrode assembly secured relative to the second jaw body, the electrode assembly including:
(1) a second electrode surface positioned to face the first electrode surface when the first and second jaws are placed in the closed configuration, the first and second electrode surfaces being operable to apply RF energy to tissue, and
(3) at least one electrical insulator interposed between the first electrode surface and the second electrode surface, the at least one electrical insulator being configured to separate the first electrode surface from the second electrode surface when in the closed configuration, the at least one electrical insulator being rigidly affixed relative to the second jaw body.

2. The surgical instrument of claim 1, wherein the at least one electrical insulator is raised away from the second electrode surface and toward the first electrode surface.

3. The surgical instrument of claim 1, wherein the at least one electrical insulator is securely affixed to the second electrode surface.

4. The surgical instrument of claim 1, wherein the at least one electrical insulator is configured to restrict a movement of the tissue between the first and second electrode surfaces when in the closed configuration.

5. The surgical instrument of claim 1, wherein the first electrode surface includes at least one recess, wherein the at least one electrical insulator is sized to nest within the at least one recess when the first and second jaws are placed in the closed configuration.

6. The surgical instrument of claim 5, wherein the at least one recess has a different shape than the at least one electrical insulator.

7. The surgical instrument of claim 1, wherein the at least one electrical insulator includes a first electrical insulator and a second electrical insulator, wherein the first electrical insulator is distally positioned relative to the first electrical insulator.

8. The surgical instrument of claim 7, wherein at least one of the first electrode surface and the second electrode surface includes a tissue grasper positioned between the first and second electrical insulators.

9. The surgical instrument of claim 8, wherein the tissue grasper protrudes from the second electrode surface less than the first electrical insulator protrudes from the second electrode surface.

10. The surgical instrument of claim 9, wherein the tissue grasper is electrically conductive.

11. The surgical instrument of claim 1, wherein the second jaw further includes an insulating spacer positioned on the second electrode surface opposite from the first electrode surface, and wherein the insulating spacer and the at least one electrical insulator are integrally formed.

12. The surgical instrument of claim 11, wherein the second jaw further includes a channel affixed to the insulating spacer such that the insulating spacer is positioned between the channel and the first electrode surface.

13. The surgical instrument of claim 1, wherein the at least one electrical insulator includes a ceramic material.

14. The surgical instrument of claim 1, wherein the at least one electrical insulator is affixed along a flat portion the second electrode surface such that the flat portion extends beyond the at least one electrical insulator.

15. The surgical instrument of claim 1, wherein the at least one electrical insulator is configured to contact the first electrode surface.

16. A surgical instrument, comprising:
(a) a shaft assembly having a distal end; and
(b) an end effector at the distal end of the shaft assembly, the end effector including:
(i) a first jaw, including:
(A) a first jaw body, and
(B) a first electrode surface secured relative to the first jaw body, and
(ii) a second jaw pivotably coupled with the first jaw and configured to selectively move from an open configuration toward a closed configuration, the second jaw including:
(A) a second jaw body having a distal end, and
(B) an electrode assembly coupled with the second jaw body, the electrode assembly including:
(1) a second electrode surface positioned to face the first electrode surface when the first and second jaws are placed in the closed configuration, the first and second electrode surfaces being operable to apply RF energy to tissue, and
(2) at least one electrical insulator interposed between the first electrode surface and the second electrode surface, the at least one electrical insulator being configured to separate the first electrode surface from the second electrode surface when in the closed configuration, the at least one electrical insulator being rigidly affixed relative to the second jaw body.

17. A method of assembling a surgical instrument, comprising:
(a) pivotally securing a first jaw to a second jaw, the first jaw including a first electrode surface, the second jaw including a second electrode surface, the first and second electrode surfaces being operable to apply RF energy to tissue;
(b) securing at least one electrical insulator along the second electrode surface; and
(c) separating the first and second electrode surfaces from each other via at least one electrical insulator.

18. The method of claim 17, further comprising nesting the at least one electrical insulator within an at least one recess of the first electrode surface.

19. The method of claim 18, further comprising forming the at least one recess using a coining and/or photo-chemical machining process.

20. The method of claim 17, further comprising grasping the tissue with the at least one electrical insulator.
